(19) United States
(12) Patent Application Publication
(10) Pub. No.: US 2022/0380316 A1
(43) Pub. Date: Dec. 1, 2022

(54) SOLID STATE FORMS OF ARRY-797 AND PROCESS FOR PREPARATION THEREOF

(71) Applicant: TEVA PHARMACEUTICALS INTERNATIONAL GMBH, Jona (CH)

(72) Inventors: Dijana Skalec Samec, Jastrebarsko (HR); Valentina Travancic, Zagreb (HR)

(21) Appl. No.: 17/772,220

(22) PCT Filed: Oct. 28, 2020
(86) PCT No.: PCT/US20/57607

§ 371 Date: Apr. 27, 2022

Related U.S. Application Data
(60) Provisional application No. 63/013,649, filed on Apr. 22, 2020.
(60) Provisional application No. 62/985,357, filed on Mar. 5, 2020.
(60) Provisional application No. 62/952,562, filed on Dec. 23, 2019.
(60) Provisional application No. 62/939,745, filed on Nov. 25, 2019.
(60) Provisional application No. 62/926,615, filed on Oct. 28, 2019.

(51) Int. Cl. C07D 231/56

(52) U.S. Cl. CPC C07D 231/56; C07B 2200/13

(57) ABSTRACT

The present disclosure encompasses solid state forms of Arry-797, in embodiments crystalline polymorphs or salts or co-crystals of Arry-797, processes for preparation thereof, and pharmaceutical compositions thereof.

### FIELD OF THE DISCLOSURE

The present disclosure encompasses solid state forms of Arry-797, in embodiments crystalline polymorphs or salts or co-crystals of Arry-797, processes for preparation thereof, and pharmaceutical compositions thereof.

### BACKGROUND OF THE DISCLOSURE

Arry-797, 5-(2,4-Difluoro-phenoxy)-1-isobutyl-1H-indazole-6-carboxylic acid (2-dimethylamino-ethyl)-amide, has the following chemical structure:

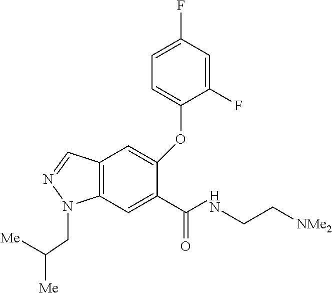 [SMILES: CC(C)Cn1ncc2cc(Oc3ccc(F)cc3F)c(C(=O)NCCN(C)C)cc21]

Arry-797, also known as Arry-371797, is reported to be a P38 mitogen-activated protein kinase inhibitors, and it is developed for the treatment of Symptomatic Dilated Cardiomyopathy Due to a Lamin A/C Gene Mutation (LMNA-related DCM).

The compound is described in International Publication No. WO2004078116.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Arry-797.

### SUMMARY OF THE DISCLOSURE

The present disclosure provides crystalline polymorphs of Arry-797, including salts and cocrystals of Arry-797, processes for preparation thereof, and pharmaceutical compositions thereof. These crystalline polymorphs can be used to prepare other solid state forms of Arry-797, cocrystals of Arry-797, Arry-797 salts and their solid state forms.

The present disclosure also provides uses of the said solid state forms of Arry-797 in the preparation of other solid state forms of Arry-797 or salts thereof.

The present disclosure provides crystalline polymorphs of Arry-797 for use in medicine, including for the treatment of LMNA-related DCM.

The present disclosure also encompasses the use of crystalline polymorphs of Arry-797 of the present disclosure for the preparation of pharmaceutical compositions and/or formulations.

In another aspect, the present disclosure provides pharmaceutical compositions comprising crystalline polymorphs of Arry-797 according to the present disclosure.

The present disclosure includes processes for preparing the above mentioned pharmaceutical compositions. The processes include combining any one or a combination of the crystalline polymorphs of Arry-797 with at least one pharmaceutically acceptable excipient.

The crystalline polymorph of Arry-797 as defined herein and the pharmaceutical compositions or formulations of the crystalline polymorph of Arry-797 may be used as medicaments, such as for the treatment of LMNA-related DCM.

The present disclosure also provides methods of treating LMNA-related DCM, by administering a therapeutically effective amount of any one or a combination of the crystalline polymorphs of Arry-797 of the present disclosure, or at least one of the above pharmaceutical compositions, to a subject suffering from LMNA-related DCM, or otherwise in need of the treatment.

The present disclosure also provides uses of crystalline polymorphs of Arry-797 of the present disclosure, or at least one of the above pharmaceutical compositions, for the manufacture of medicaments for treating e.g. LMNA-related DCM.

### BRIEF DESCRIPTION OF THE DRAWINGS

FIG. 1 shows a characteristic X-ray powder diffraction pattern (XRPD) of Arry-797 Form 1.

FIG. 2 shows a characteristic XRPD of Crystalline Form M1 of Arry-797: maleic acid complex (Arry-797 maleate).

FIG. 3 shows a characteristic XRPD of Crystalline Form F1 of Arry-797: fumaric acid complex (Arry-797 fumarate).

FIG. 4 shows a characteristic XRPD of Crystalline Form F2 of Arry-797: fumaric acid complex (Arry-797 fumarate).

FIG. 5 shows a characteristic XRPD of Crystalline Form T1 of Arry-797: tartaric acid complex (Arry-797 tartrate).

FIG. 6 shows a characteristic XRPD of Crystalline Form H1 of Arry-797 HCl.

FIG. 7 shows a characteristic XRPD of Crystalline Form F3 of Arry-797 fumarate.

FIG. 8 shows a characteristic XRPD of a mixture of form F1 and F4 Arry-797 fumarate.

FIG. 9 shows a characteristic XRPD of Crystalline Form F5 of Arry-797 fumarate.

FIG. 10 shows a characteristic XRPD of Crystalline Form P1 of Arry-797: palmitic acid complex.

### DETAILED DESCRIPTION OF THE DISCLOSURE

The present disclosure encompasses solid state forms of Arry-797, including crystalline polymorphs of Arry-797, processes for preparation thereof, and pharmaceutical compositions thereof.

Solid state properties of Arry-797 and crystalline polymorphs thereof can be influenced by controlling the conditions under which Arry-797 and crystalline polymorphs thereof are obtained in solid form.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Arry-797 described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Arry-797. In some embodiments of the disclosure, the described crystalline polymorph of Arry-797 may contain from about 1% to about 20% (w/w), from about 5% to about 20% (w/w), or from about 5% to about 10% (w/w) of one or more other crystalline polymorph of the same Arry-797.

In some embodiments of the disclosure, the described crystalline polymorph of Arry-797 may contain no more than about 20%, no more than about 10%, no more than about 5%, no more than about 2%, no more than about 1% or about 0% of amorphous form of Arry-797. Depending on which other crystalline polymorphs a comparison is made, the crystalline polymorphs of Arry-797 of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Arry-797 referred to herein as being characterized by graphical data “as depicted in” or “as substantially depicted in” a Figure will thus be understood to include any crystal forms of Arry-797 characterized with the graphical data having such small variations, as are well known to the skilled person, in comparison with the Figure.

As used herein, and unless stated otherwise, the term “anhydrous” in relation to crystalline forms of Arry-797, relates to a crystalline form of Arry-797 which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, the term “isolated” in reference to crystalline polymorph of Arry-797 of the present disclosure corresponds to a crystalline polymorph of Arry-797 that is physically separated from the reaction mixture in which it is formed.

“Co-Crystal” or “Cocrystal” as used herein is defined as a crystalline material including two or more molecules in the same crystalline lattice and associated by non-ionic and non-covalent bonds. In some embodiments, the cocrystal includes two molecules which are in natural state.

“Cocrystal former” or “crystal former” as used herein is defined as a molecule that forms a cocrystal with Arry-797 or salts thereof, for example maleic acid, fumaric acid and/or tartaric acid.

As used herein, crystalline Arry-797: maleic acid is a distinct molecular species. Crystalline Arry-797: maleic acid may be a co-crystal of Arry-797 and maleic acid. Alternatively, crystalline Arry-797: maleic acid may be a Arry-797 maleate salt.

As used herein, crystalline Arry-797: fumaric acid is a distinct molecular species. Crystalline Arry-797: fumaric acid may be a co-crystal of Arry-797 and fumaric acid. Alternatively, crystalline Arry-797: fumaric acid may be a Arry-797 fumarate salt.

As used herein, crystalline Arry-797: tartaric acid is a distinct molecular species. Crystalline Arry-797: tartaric acid may be a co-crystal of Arry-797 and tartaric acid. Alternatively, crystalline Arry-797: tartaric acid may be a Arry-797 tartarate salt.

As used herein, crystalline Arry-797: palmitic acid is a distinct molecular species. Crystalline Arry-797: palmitic acid may be a co-crystal of Arry-797 and palmitic acid. Alternatively, crystalline Arry-797: palmitic acid may be a Arry-797 palmitate salt.

As used herein, solid state forms of Arry-797 and Arry-797 salts include co-crystal forms of Arry-797. Preferably, the solid state forms of Arry-797 with maleic acid, fumaric acid, tartaric acid, and hydrochloric acid disclosed herein are salts (i.e. Arry-797 maleate, Arry-797 fumarate, Arry-797 tartrate, and Arry-797 hydrochloride).

As used herein, crystalline polymorph of Arry-797 includes co-crystal forms of Arry-797 and crystalline forms of Arry-797 salts.

As used herein, unless stated otherwise, the XRPD measurements are taken using copper Kα radiation wavelength 1.54187 Å. XRPD peaks reported herein are measured using CuK α radiation, λ=1.54187 Å, typically at a temperature of 25±3° C.

As used herein, unless stated otherwise, ¹³C NMR reported herein are measured at 125 MHz at a magic angle spinning frequency ωᵣ/2π=11 kHz, preferably at a temperature of at 293 K±3° C.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, unless stated otherwise, crystals filtration refers to vacuum filtration.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

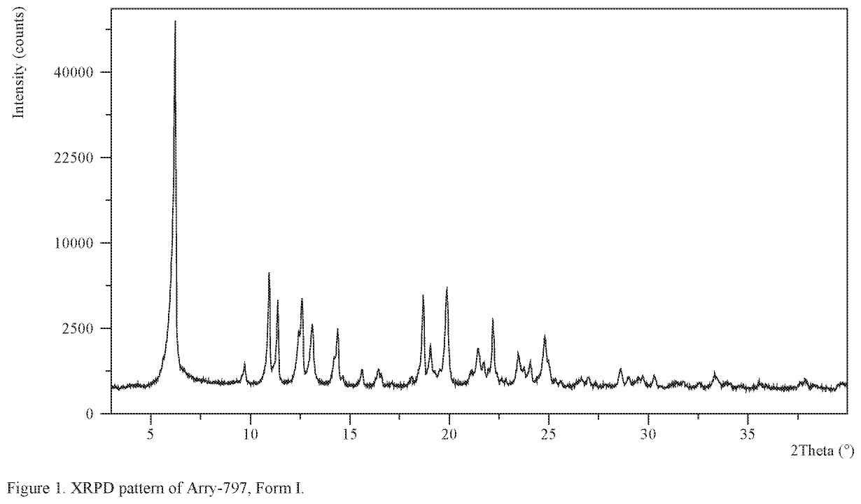

The present disclosure includes a crystalline polymorph of Arry-797, designated Form 1. The crystalline Form 1 of Arry-797 may be characterized by data selected from one or more of the following: an X-ray powder diffraction pattern substantially as depicted in FIG. 1; an X-ray powder diffraction pattern having peaks at 6.2, 10.9, 11.4, 18.7 and 19.9 degrees 2-theta±0.2 degrees 2-theta; and combinations of these data.

Crystalline Form 1 of Arry-797 may be further characterized by an X-ray powder diffraction pattern having peaks at 6.2, 10.9, 11.4, 18.7 and 19.9 degrees 2-theta±0.2 degrees 2-theta, and also having any one, two, three, four or five additional peaks selected from 12.6, 13.1, 14.4, 22.2 and 24.8 degrees 2-theta±0.2 degrees 2-theta.

Crystalline Form 1 of Arry-797 may be alternatively characterized by an X-ray powder diffraction pattern having peaks at 6.2, 10.9, 11.4, 12.6, 13.1, 14.4, 18.7, 19.9, 22.2, and 24.8 degrees 2-theta±0.2 degrees 2-theta.

In one embodiment of the present disclosure, crystalline Form 1 of Arry-797 is isolated.

Crystalline Form 1 of Arry-797 may be anhydrous form.

Crystalline Form 1 of Arry-797 may be characterized by each of the above characteristics alone/or by all possible combinations, e.g., an XRPD pattern having peaks at 6.2, 10.9, 11.4, 18.7 and 19.9 degrees 2-theta±0.2 degrees 2-theta; an XRPD pattern as depicted in FIG. 1, and combinations thereof.

The present disclosure further encompasses a crystalline complex of Arry-797 and maleic acid. Crystalline Arry-797: maleic acid complexes may be a co-crystal of Arry-797 and maleic acid. Alternatively, crystalline Arry-797: maleic acid may be a salt, i.e., Arry-797 maleate.

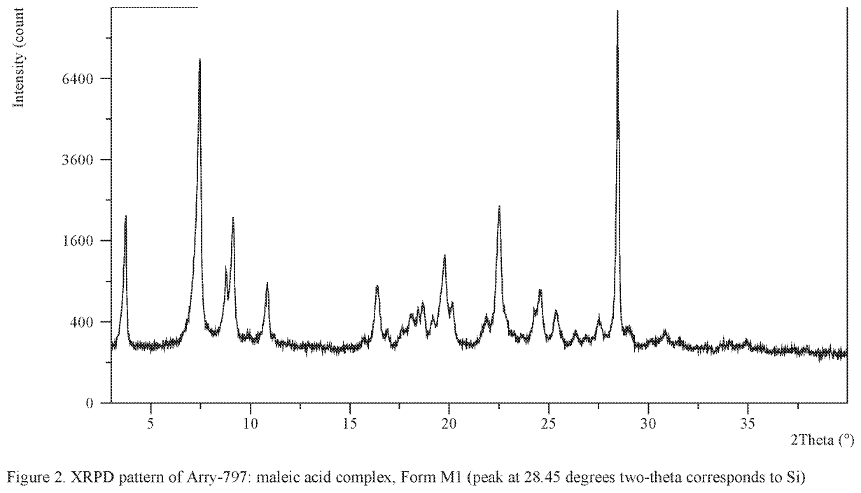

The disclosure further encompasses a crystalline complex of Arry-797 and maleic acid, or particularly a crystalline form of Arry-797 maleate, designated Form M1. Crystalline Form M1 of Arry-797: maleic acid, particularly crystalline form M1 of Arry-797 maleate, may be characterized by data selected from one or more of the following: an X-ray powder diffraction pattern substantially as depicted in FIG. 2; an X-ray powder diffraction pattern having peaks at 3.7, 7.5, 9.2, 22.5 and 24.6 degrees 2-theta±0.2 degrees 2-theta; and combinations of these data.

Crystalline Form M1 of Arry-797: maleic acid complex, particularly crystalline Form M1 of Arry-797 maleate, may be further characterized by an X-ray powder diffraction pattern having peaks at 3.7, 7.5, 9.2, 22.5 and 24.6 degrees 2-theta±0.2 degrees 2-theta, and also having any one, two, three or four additional peaks selected from 19.8, 21.9, 25.4 and 26.3 degrees 2-theta±0.2 degrees 2-theta.

Crystalline Form M1 of Arry-797: maleic acid complex, particularly crystalline Form M1 of Arry-797 maleate, may be alternatively characterized by an X-ray powder diffraction pattern having peaks at 3.7, 7.5, 9.2, 19.8, 21.9, 22.5, 24.6, 25.4 and 26.3 degrees 2-theta±0.2 degrees 2-theta.

Preferably, in any embodiment disclosed herein, crystalline Form M1 is a salt, i.e. Arry-797 maleate.

In embodiments of the present disclosure, crystalline Form M1 of Arry-797: maleic acid complex, particularly crystalline Form M1 of Arry-797 maleate, is isolated.

In embodiments of the present disclosure, crystalline Form M1 of Arry-797: maleic acid complex, particularly crystalline Form M1 of Arry-797 maleate, may be anhydrous form.

Crystalline Form M1 of Arry-797: maleic acid complex, particularly crystalline Form M1 of Arry-797 maleate, may be characterized by each of the above characteristics alone or by all possible combinations, e.g., an XRPD pattern having peaks at 3.7, 7.5, 9.2, 22.5 and 24.6 degrees 2-theta±0.2 degrees 2-theta; an XRPD pattern as depicted in FIG. 2; and combinations thereof.

The present disclosure further encompasses a crystalline complex of Arry-797 and fumaric acid. Crystalline Arry-797: fumaric acid complexes may be a co-crystal of Arry-797 and fumaric acid. Alternatively, crystalline Arry-797: fumaric acid may be a salt, i.e., Arry-797 fumarate.

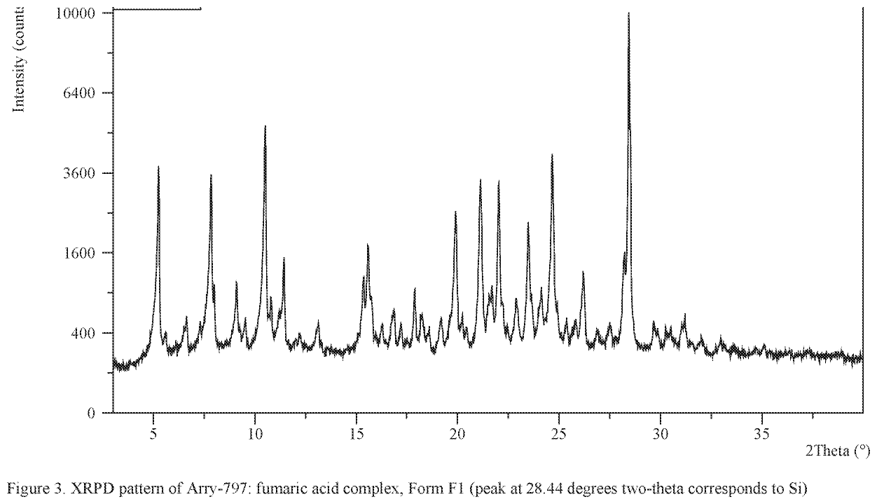

The disclosure further encompasses a crystalline complex of Arry-797 and fumaric acid, particularly crystalline Form F1 of Arry-797 fumarate, designated Form F1. Crystalline Form F1 of Arry-797: fumaric acid, particularly crystalline Form F1 of Arry-797 fumarate, may be characterized by data selected from one or more of the following: an X-ray powder diffraction pattern substantially as depicted in FIG. 3; an X-ray powder diffraction pattern having peaks at 5.3, 7.9, 10.5 and 16.9 degrees 2-theta±0.2 degrees 2-theta; and combinations of these data.

Crystalline Form F1 of Arry-797: fumaric acid complex, particularly crystalline Form F1 of Arry-797 fumarate, may be further characterized by an X-ray powder diffraction pattern having peaks at 5.3, 7.9, 10.5 and 16.9 degrees 2-theta±0.2 degrees 2-theta, and also having any one, two, three or four additional peaks selected from 9.1, 11.5, 19.9 and 21.1 degrees 2-theta±0.2 degrees 2-theta.

Crystalline Form F1 of Arry-797: fumaric acid complex, particularly crystalline Form F1 of Arry-797 fumarate, may be alternatively characterized by an X-ray powder diffraction pattern having peaks at 5.3, 7.9, 9.1, 10.5, 11.5, 16.9, 19.9 and 21.1 degrees 2-theta±0.2 degrees 2-theta.

Preferably, in any embodiment disclosed herein, crystalline Form F1 is a salt, i.e. Arry-797 fumarate.

In embodiments of the present disclosure, crystalline Form F1 of Arry-797: fumaric acid complex, particularly crystalline Form F1 of Arry-797 fumarate, is isolated.

In embodiments of the present disclosure, crystalline Form F1 of Arry-797: fumaric acid complex, particularly crystalline Form F1 of Arry-797 fumarate, may be anhydrous form.

Crystalline Form F1 of Arry-797: fumaric acid complex, particularly crystalline Form F1 of Arry-797 fumarate, may be characterized by each of the above characteristics alone or by all possible combinations, e.g., an XRPD pattern having peaks at 5.3, 7.9, 10.5 and 16.9 degrees 2-theta±0.2 degrees 2-theta; an XRPD pattern as depicted in FIG. 3; and combinations thereof.

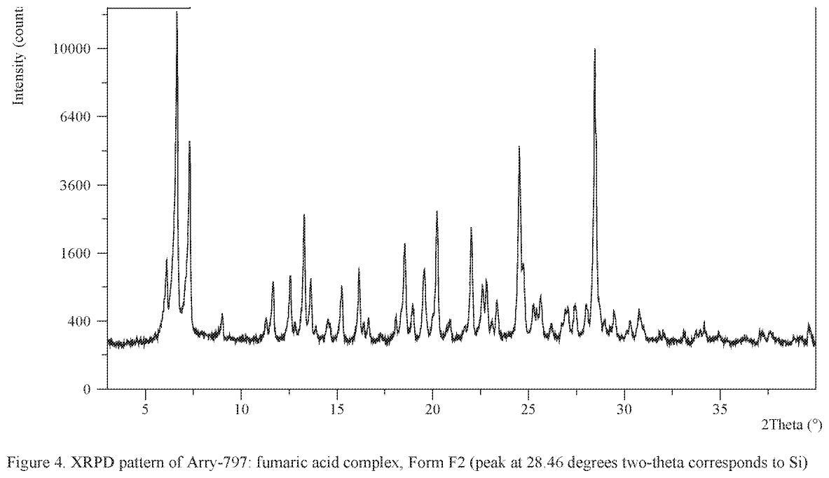

The disclosure further encompasses a crystalline complex of Arry-797 and fumaric acid, particularly crystalline Arry-797 fumarate, designated Form F2. Crystalline Form F2 of Arry-797: fumaric acid, particularly crystalline Form F2 of Arry-797 fumarate, may be characterized by data selected from one or more of the following: an X-ray powder diffraction pattern substantially as depicted in FIG. 4; an X-ray powder diffraction pattern having peaks at 6.6, 7.3, 13.3, 16.1, and 20.2 degrees 2-theta±0.2 degrees 2-theta; and combinations of these data.

Crystalline Form F2 of Arry-797: fumaric acid complex, particularly crystalline Form F2 of Arry-797 fumarate, may be further characterized by an X-ray powder diffraction pattern having peaks at 6.6, 7.3, 13.3, 16.1, and 20.2 degrees 2-theta±0.2 degrees 2-theta, and also having any one, two, three, four or five additional peaks selected from 11.7, 15.3, 19.6, 22.0 and 24.5 degrees 2-theta±0.2 degrees 2-theta.

Crystalline Form F2 of Arry-797: fumaric acid complex, particularly crystalline Form F2 of Arry-797 fumarate, may be alternatively characterized by an X-ray powder diffraction pattern having peaks at 6.6, 7.3, 11.7, 13.3, 15.3, 16.1, 19.6, 20.2, 22.0, and 24.5 degrees 2-theta±0.2 degrees 2-theta.

Preferably, in any embodiment disclosed herein, crystalline Form F2 is a salt, i.e. Arry-797 fumarate.

In embodiments of the present disclosure, crystalline Form F2 of Arry-797: fumaric acid complex, particularly crystalline Form F2 of Arry-797 fumarate, is isolated.

In embodiments of the present disclosure, crystalline Form F2 of Arry-797: fumaric acid complex, particularly crystalline Form F2 of Arry-797 fumarate, may be a hydrate form.

Crystalline Form F2 of Arry-797: fumaric acid complex, particularly crystalline Form F2 of Arry-797 fumarate, may be characterized by each of the above characteristics alone or by all possible combinations, e.g., an XRPD pattern having peaks at 6.6, 7.3, 13.3, 16.1, and 20.2 degrees 2-theta±0.2 degrees 2-theta; an XRPD pattern as depicted in FIG. 4; and combinations thereof.

The present disclosure further encompasses a crystalline complex of Arry-797 and tartaric acid, particularly crystalline Arry-797 tartrate. Crystalline Arry-797: tartaric acid complexes may be a co-crystal of Arry-797 and tartaric acid. Alternatively, crystalline Arry-797: tartaric acid may be a salt, i.e., Arry-797 tartrate.

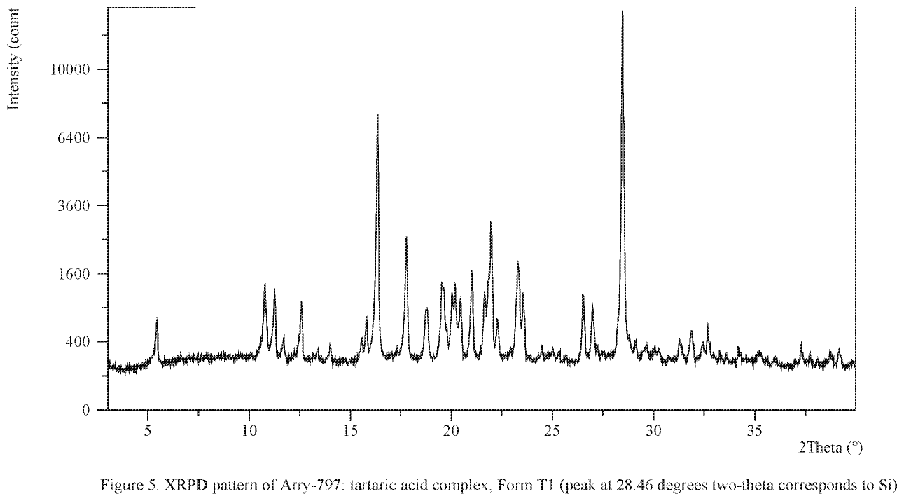

The disclosure further encompasses a crystalline complex of Arry-797 and tartaric acid, particularly crystalline Arry-797 tartrate, designated Form T1. Crystalline Form T1 of Arry-797: tartaric acid, particularly crystalline Form T1 of Arry-797 tartrate, may be characterized by data selected from one or more of the following: an X-ray powder diffraction pattern substantially as depicted in FIG. 5; an X-ray powder diffraction pattern having peaks at 5.4, 16.4, 17.8 and 22.0 degrees 2-theta±0.2 degrees 2-theta; and combinations of these data.

Crystalline Form T1 of Arry-797: tartaric acid complex, particularly crystalline form T1 of Arry-797 tartrate, may be further characterized by an X-ray powder diffraction pattern having peaks at 5.4, 16.4, 17.8 and 22.0 degrees 2-theta±0.2 degrees 2-theta, and also having any one, two, three or four additional peaks selected from 12.6, 19.6, 21.0 and 26.5 degrees 2-theta±0.2 degrees 2-theta.

Crystalline Form T1 of Arry-797: tartaric acid complex, particularly crystalline Form T1 of Arry-797 tartrate, may be alternatively characterized by an X-ray powder diffraction pattern having peaks at 5.4, 12.6, 16.4, 17.8, 19.6, 21.0, 22.0, and 26.5 degrees 2-theta±0.2 degrees 2-theta. Preferably, in any embodiment disclosed herein, crystalline Form T1 is a salt, i.e. Arry-797 tartrate.

In embodiments of the present disclosure, crystalline Form T1 of Arry-797: tartaric acid complex, particularly crystalline Form T1 of Arry-797 tartrate, is isolated.

In embodiments of the present disclosure, crystalline Form T1 of Arry-797: tartaric acid complex, particularly crystalline Form T1 of Arry-797 tartrate, may be anhydrous form.

Crystalline Form T1 of Arry-797: tartaric acid complex, particularly crystalline Form T1 of Arry-797 tartrate, may be characterized by each of the above characteristics alone or by all possible combinations, e.g., an XRPD pattern having peaks at 5.4, 16.4, 17.8 and 22.0 degrees 2-theta±0.2 degrees 2-theta; an XRPD pattern as depicted in FIG. 5; and combinations thereof.

The present disclosure further encompasses Arry-797 hydrochloride salt. In embodiments Arry-797 HCl salt is a crystalline form.

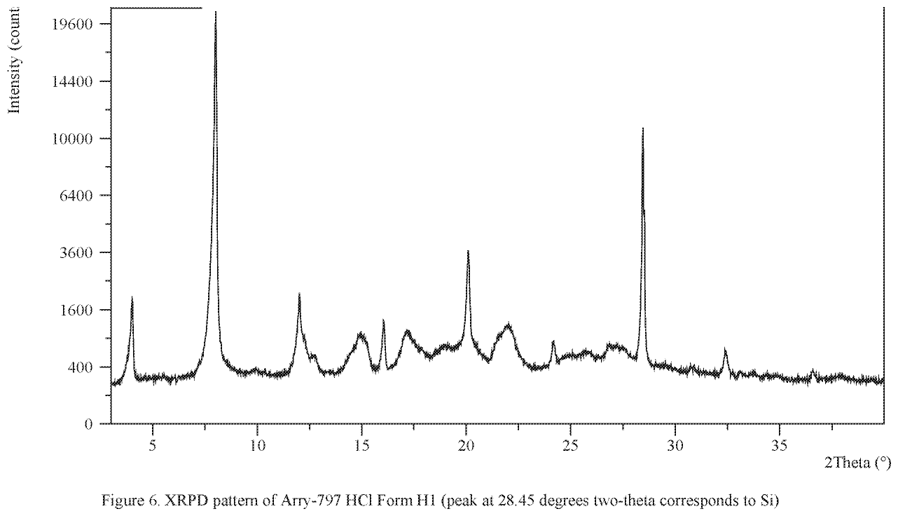

The present disclosure further encompasses a crystalline polymorph of Arry-797 HCl salt, designated Form H1. The crystalline Form H1 of Arry-797 may be characterized by data selected from one or more of the following: an X-ray powder diffraction pattern substantially as depicted in FIG. 6; an X-ray powder diffraction pattern having peaks at 4.0, 8.0, 12.0, 16.1 and 20.1 degrees 2-theta±0.2 degrees 2-theta; and combinations of these data.

Crystalline Form H1 of Arry-797 HCl salt may be further characterized by an X-ray powder diffraction pattern having peaks at 4.0, 8.0, 12.0, 16.1 and 20.1 degrees 2-theta±0.2 degrees 2-theta, and also having any one, two, three, four or five additional peaks selected from 14.9, 17.1, 22.2, 24.2 and 32.4 degrees 2-theta±0.2 degrees 2-theta.

Crystalline Form H1 of Arry-797 HCl salt may be alternatively characterized by an X-ray powder diffraction pattern having peaks at 4.0, 8.0, 12.0, 14.9, 16.1, 17.1, 20.1, 22.2, 24.2 and 32.4 degrees 2-theta±0.2 degrees 2-theta.

In one embodiment of the present disclosure, crystalline Form H1 of Arry-797 HCl salt is isolated.

In one embodiment of the present disclosure, crystalline Form H1 of Arry-797 HCl salt may be anhydrous form.

Crystalline Form H1 of Arry-797 HCl salt may be characterized by each of the above characteristics alone/or by all possible combinations, e.g., an XRPD pattern having peaks at 4.0, 8.0, 12.0, 16.1 and 20.1 degrees 2-theta±0.2 degrees 2-theta; an XRPD pattern as depicted in FIG. 6, and combinations thereof.

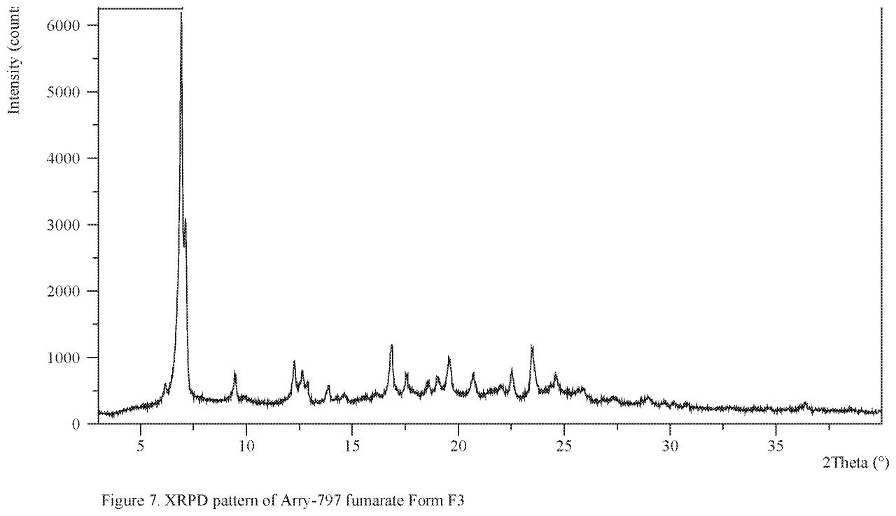

The disclosure further encompasses a crystalline polymorph of Arry-797 fumarate salt, designated Form F3. Crystalline Form F3 of Arry-797 fumarate may be characterized by data selected from one or more of the following: an X-ray powder diffraction pattern substantially as depicted in FIG. 7; an X-ray powder diffraction pattern having peaks at 6.9, 12.3, 12.5 and 22.5 degrees 2-theta±0.2 degrees 2-theta; and combinations of these data.

Crystalline Form F3 of Arry-797 fumarate may be further characterized by an X-ray powder diffraction pattern having peaks at 6.9, 12.3, 12.5 and 22.5 degrees 2-theta±0.2 degrees 2-theta, and also having any one, two, three or four additional peaks selected from 7.1, 9.5, 17.5 and 20.7 degrees 2-theta±0.2 degrees 2-theta.

Crystalline Form F3 of Arry-797 fumarate may be alternatively characterized by an X-ray powder diffraction pattern having peaks at 6.9, 7.1, 9.5, 12.3, 12.5, 17.5, 20.7 and 22.5 degrees 2-theta±0.2 degrees 2-theta.

In embodiments of the present disclosure, crystalline Form F3 of Arry-797 fumarate is isolated.

Crystalline Form F3 of Arry-797 fumarate may be characterized by each of the above characteristics alone or by all possible combinations, e.g., an XRPD pattern having peaks at 6.9, 12.3, 12.5 and 22.5 degrees 2-theta±0.2 degrees 2-theta; an XRPD pattern as depicted in FIG. 7; and combinations thereof.

The disclosure further encompasses a crystalline polymorph of Arry-797 fumarate salt, designated Form F4. Crystalline Form F4 of Arry-797 fumarate may be characterized by an X-ray powder diffraction pattern having peaks at 8.0, 9.3, and 12.0 degrees 2-theta±0.2 degrees 2-theta.

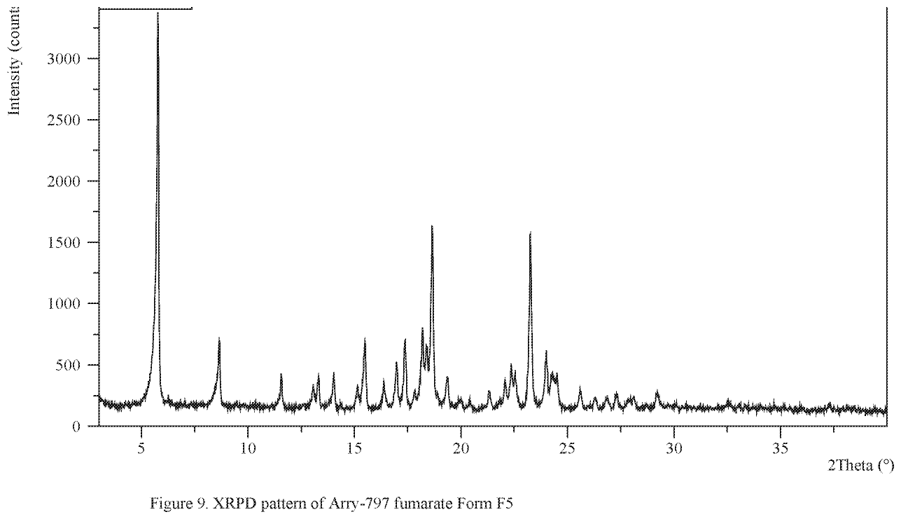

The disclosure further encompasses a crystalline polymorph of Arry-797 fumarate salt, designated Form F5. Crystalline Form F5 of Arry-797 fumarate may be characterized by data selected from one or more of the following: an X-ray powder diffraction pattern substantially as depicted in FIG. 9; an X-ray powder diffraction pattern having peaks at 5.8, 8.7, 14.0, 17.4, 19.4 and 23.3 degrees 2-theta±0.2 degrees 2-theta; and combinations of these data.

Crystalline Form F5 of Arry-797 fumarate may be further characterized by an X-ray powder diffraction pattern having peaks at 5.8, 8.7, 14.0, 17.4, 19.4 and 23.3 degrees 2-theta±0.2 degrees 2-theta, and also having any one, two, three, four or five additional peaks selected from 11.6, 13.3, 15.5, 17.4 and 18.7 degrees 2-theta±0.2 degrees 2-theta.

Alternatively, crystalline Form F5 of Arry-797 fumarate may be characterized by data selected from one or more of the following: an X-ray powder diffraction pattern substantially as depicted in FIG. 9; an X-ray powder diffraction pattern having peaks at 5.8, 8.7, 14.0, 19.4 and 23.3 degrees 2-theta±0.2 degrees 2-theta; and combinations of these data. Crystalline Form F5 of Arry-797 fumarate may be alternatively characterized by an X-ray powder diffraction pattern having peaks at 5.8, 8.7, 14.0, 19.4 and 23.3 degrees 2-theta±0.2 degrees 2-theta, and also having any one, two, three, four or five additional peaks selected from 11.6, 13.3, 15.5, 17.4 and 18.7 degrees 2-theta±0.2 degrees 2-theta.

Crystalline Form F5 of Arry-797 fumarate may be alternatively characterized by an X-ray powder diffraction pattern having peaks at 5.8, 8.7, 11.6, 13.3, 14.0, 15.5, 17.4, 18.7, 19.4 and 23.3 degrees 2-theta±0.2 degrees 2-theta.

In embodiments of the present disclosure, crystalline Form F5 of Arry-797 fumarate is isolated.

In embodiments of the present disclosure, crystalline Form F5 of Arry-797 fumarate may be anhydrous form.

Crystalline Form F5 of Arry-797 fumarate may be characterized by each of the above characteristics alone or by all possible combinations, e.g., an XRPD pattern having peaks at 5.8, 8.7, 14.0, 17.4, 19.4 and 23.3 degrees 2-theta±0.2 degrees 2-theta; an XRPD pattern as depicted in FIG. 9; and combinations thereof, or an XRPD pattern having peaks at 5.8, 8.7, 14.0, 19.4 and 23.3 degrees 2-theta±0.2 degrees 2-theta; an XRPD pattern as depicted in FIG. 9; and combinations thereof.

The present disclosure further encompasses a crystalline complex of Arry-797 and palmitic acid. Crystalline Arry-797: palmitic acid complexes may be a co-crystal of Arry-797 and palmitic acid. Alternatively, crystalline Arry-797: palmitic acid may be a salt, i.e., Arry-797 palmitate.

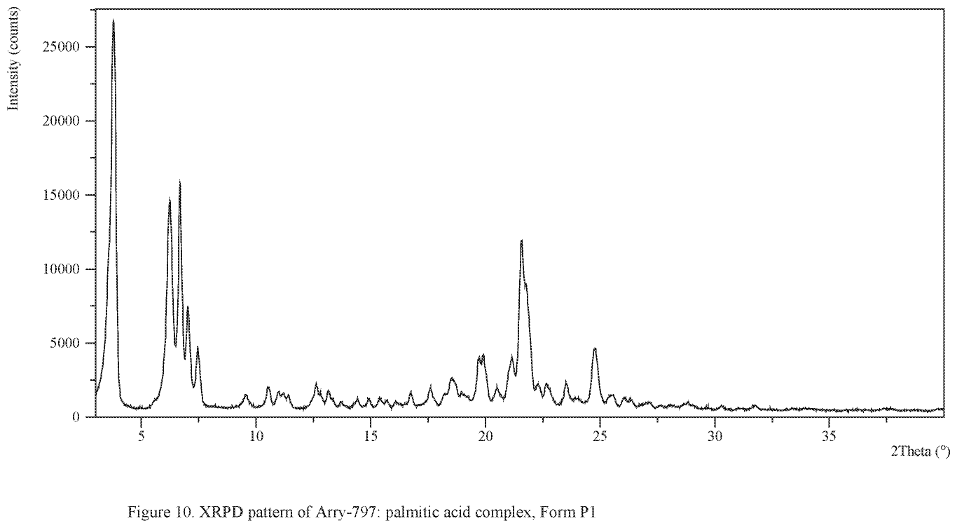

The disclosure further encompasses a crystalline complex of Arry-797 and palmitic acid, designated Form P1. Crystalline Form P1 of Arry-797: palmitic acid may be characterized by data selected from one or more of the following: an X-ray powder diffraction pattern substantially as depicted in FIG. 10; an X-ray powder diffraction pattern having peaks at 3.9, 6.7, 7.0, 7.5 and 10.6 degrees 2-theta±0.2 degrees 2-theta; and combinations of these data.

Crystalline Form P1 of Arry-797: palmitic acid complex may be further characterized by an X-ray powder diffraction pattern having peaks at 3.9, 6.7, 7.0, 7.5 and 10.6 degrees 2-theta±0.2 degrees 2-theta, and also having any one, two, three, four or five additional peaks selected from 9.6, 17.6, 20.5, 21.6 and 22.7 degrees 2-theta±0.2 degrees 2-theta.

Crystalline Form P1 of Arry-797: palmitic acid complex may be further characterized by an X-ray powder diffraction pattern having peaks at 3.9, 6.7, 7.0, 7.5, 9.6, 10.6, 17.6, 20.5, 21.6 and 22.7 degrees 2-theta±0.2 degrees 2-theta.

In embodiments of the present disclosure, crystalline Form P1 of Arry-797: palmitic acid complex is isolated.

In embodiments of the present disclosure, crystalline Form P1 of Arry-797: palmitic acid complex may be anhydrous form.

Crystalline Form P1 of Arry-797: palmitic acid complex may be characterized by each of the above characteristics alone or by all possible combinations, e.g., an XRPD pattern having peaks at 3.9, 6.7, 7.0, 7.5 and 10.6 degrees 2-theta±0.2 degrees 2-theta; an XRPD pattern as depicted in FIG. 10; and combinations thereof.

The above crystalline polymorphs can be used to prepare other crystalline polymorphs of Arry-797, Arry-797 salts and their solid state forms.

The present disclosure encompasses a process for preparing other solid state forms of Arry-797, Arry-797 salts and their solid state forms thereof. The process includes preparing any one of the Arry-797 (salts) and solid state forms of Arry-797 by the processes of the present disclosure, and converting it to other Arry-797 salt. Alternatively, the process includes preparing any one of the crystalline polymorphs of Arry-797 of the present invention, by the processes of the present disclosure, and converting it to another Arry-797 form. The conversion can be done, for example, by a process including basifying any one or a combination of the above described Arry-797 (salts) and/or solid state forms thereof, and reacting the obtained Arry-797 base with an appropriate acid, to obtain the corresponding salt. Alternatively, the conversion can be done by salt switching, i.e., reacting a Arry-797 acid addition salt, with an acid having a pKa which is lower than the pKa of the acid of the first Arry-797 acid addition salt.

The present disclosure provides the above described crystalline polymorphs of Arry-797 for use in the preparation of pharmaceutical compositions comprising Arry-797 and/or crystalline polymorphs thereof.

The present disclosure also encompasses the use of crystalline polymorphs of Arry-797 of the present disclosure for the preparation of pharmaceutical compositions of crystalline polymorph Arry-797 and/or crystalline polymorphs thereof.

The present disclosure includes processes for preparing the above mentioned pharmaceutical compositions. The processes include combining any one or a combination of the crystalline polymorphs of Arry-797 of the present disclosure with at least one pharmaceutically acceptable excipient.

Pharmaceutical combinations or formulations of the present disclosure contain any one or a combination of the solid state forms of Arry-797 of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Arry-797 and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Arry-797 can be administered. Arry-797 may be formulated for administration to a mammal, in embodiments to a human, by injection. Arry-797 can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Arry-797 and the pharmaceutical compositions and/or formulations of Arry-797 of the present disclosure can be used as medicaments, in embodiments in the treatment of LMNA-related DCM.

The present disclosure also provides methods of treating LMNA-related DCM by administering a therapeutically effective amount of any one or a combination of the crystalline polymorphs of Arry-797 of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

Sample after being powdered in a mortar and pestle is applied directly on a silicon plate holder. The X-ray powder diffraction pattern was measured with Philips X'Pert PRO X-ray powder diffractometer, equipped with Cu irradiation source=1.54184 \acute{Å} (\acute{Å}ngstrom), X'Celerator (2.022° 2θ) detector. Scanning parameters: angle range: 3-40 deg., step size 0.0167, time per step 37 s, continuous scan. The described peak positions were determined with or without using silicon powder as an internal standard. When used, the position of the silicon (Si) peak was corrected to silicone theoretical peak: 28.45 degrees two theta, and the positions of the measured peaks were corrected respectively.

### EXAMPLES

### Preparation of Starting Materials

Arry-797 can be prepared according to methods known from the literature, for example International Publication No. WO 2004/078116. Preferably, in Examples 4, 5, 6, 7, 8, 9, 10 and 11, the respective salt forms of Arry-797 maleate, fumarate, tartrate and hydrochloride, are prepared.

### Example 1: Preparation of Arry-797 Form 1

Arry-797 crude material (50 mg) was dissolved in 0.5 ml of acetone at room temperature. Prepared solution was left open at room conditions for solvent to evaporate. After crystallization occurred, crystals were filtrated off and analyzed by XRPD. Arry-797, Form 1, was obtained.

### Example 2: Preparation of Arry-797 Form 1

Arry-797 crude material (50 mg) was dissolved in 0.5 ml of ethyl acetate at room temperature. Prepared solution was left open at room conditions for solvent to evaporate. After crystallization occurred, crystals were filtrated off and analyzed by XRPD. Arry-797, Form 1, was obtained.

### Example 3: Preparation of Arry-797 Form 1

Arry-797 crude material (50 mg) was dissolved in 0.5 ml of tert-butanol at room temperature. Prepared solution was left open at room conditions for solvent to evaporate. After crystallization occurred, crystals were filtrated off and analyzed by XRPD. Arry-797, Form 1, was obtained.

### Example 4: Preparation of Arry-797: Maleic Acid Complex Form M1

Arry-797 crude material (500 mg) was dissolved in 15 ml of acetone at room temperature. Maleic acid (2.0 eq., 278.73 mg) was added into solution. Maleic acid was dissolved and solution was put into ice bath. After crystallization occurred, crystals were filtrated off and analyzed by XRPD. Crystalline Form M1 of Arry-797: maleic acid complex was obtained.

### Example 5: Preparation of Arry-797: Fumaric Acid Complex Form F1

Arry-797 crude material (500 mg) was dissolved in 13 ml of acetone at room temperature. Fumaric acid (2.0 eq., 278.73 mg) was added into solution. Fumaric acid was dissolved and solution was put into ice bath. After crystallization occurred, crystals were filtrated off and analyzed by XRPD. Crystalline Form F1 of Arry-797: fumaric acid complex was obtained.

### Example 6: Preparation of Arry-797: Fumaric Acid Complex Form F2

Arry-797 crude material (500 mg) was dissolved in 22 ml of a mixture acetone-water (1:1) at room temperature. Fumaric acid (2.0 eq., 278.73 mg) was added into solution. Fumaric acid was dissolved, solution was put into ice bath. After crystallization occurred, crystals were filtrated off and analyzed by XRPD. Crystalline Form F2 of Arry-797: fumaric acid complex was obtained.

### Example 7: Preparation of Arry-797: Tartaric Acid Complex Form T1

Arry-797 crude material (500 mg) was dissolved in 15 ml of acetone at room temperature. Tartaric acid (1.0 eq., 180.2 mg) was added into solution. Tartaric acid was dissolved and solution was put into ice bath. After crystallization occurred, crystals were filtrated off and analyzed by XRPD. Crystalline Form T1 of Arry-797: tartaric acid complex was obtained.

### Example 8: Preparation of Arry-797 HCl Salt Form H1

Arry-797 crude material (500 mg) was dissolved in 10 ml of 2-propanol at room temperature. Hydrochloric acid (1.0 eq., 1.24 mL, 1 M) was added into solution. Solution was cooled down to 0-5° C. After crystallization occurred, crystals were filtrated off and analyzed by XRPD. Arry-797 HCl Form H1 was obtained.

### Example 9: Preparation of Arry-797 Fumarate Form F3

Arry-797: fumaric acid complex Form F2 was dried in a vacuum dryer at 80° C. for 2 hours. After drying, the obtained solid was analyzed by XRPD. Arry-797 fumarate Form F3 was obtained.

### Example 10: Preparation of Arry-797 Fumarate Form F4

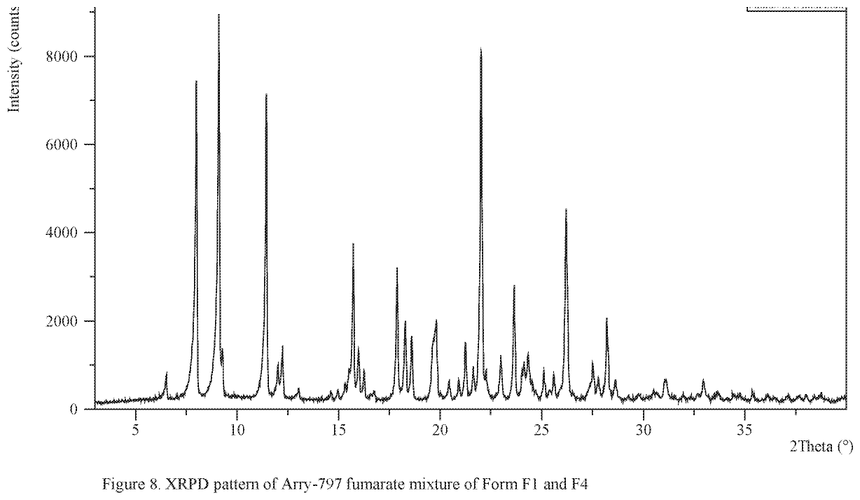

Arry-797 crude material (1000 mg) was dissolved in 20 ml of acetone at room temperature. Solution was stirred for 30 minutes and then a solution of fumaric acid (2 eq. 557.7 mg in 17 ml) was added. Solution of API and fumaric acid was stirred for 1 hour and then was put into ice bath. After crystallization occurred, crystals were filtrated off and analyzed by XRPD. Arry-797 fumarate, mixture of form F1 and F4 was obtained (FIG. 8).

### Example 11: Preparation of Arry-797 Fumarate Form F5

Arry-797: fumaric acid complex Form F2 was suspended in acetone (concentration 100 g/L) at room temperature. Suspension was vacuum filtrated after 4 hours and analyzed by XRPD. Arry-797 fumarate, Form F5, was obtained.

### Example 12: Preparation of Arry-797: Palmitic Acid Complex Form P1

Arry-797 Form I (45 mg) and palmitic acid (2.eq 55 mg) were subjected to milling in ball mill (agate jar; frequency 25 Hz; time: 30 minutes) with additional 20 μL of acetone. Sample was analyzed after 30 and 60 minutes. Arry 797: palmitic acid, Form P1, was obtained.

### Example 13: Preparation of Arry-797: Palmitic Acid Complex Form P1

Arry-797 Form I (45 mg) and palmitic acid (2.eq 55 mg) were suspended in acetone (1 ml), ethanol (1 ml) or ethyl acetate (1 ml). Suspension was dissolved immediately at RT and was left to evaporate. After 4 hours solution was crystallized. Sample was analyzed by XRPD. Arry-797: palmitic acid, Form P1 was obtained.

### Example 14: Preparation of Arry-797: Palmitic Acid Complex Form P1

Arry-797 Form I (45 mg) and palmitic acid (2.eq 55 mg) were suspended in acetone (1 ml), ethanol (1 ml) or ethyl acetate (1 ml). Suspension was dissolved immediately at RT and was left to evaporate. After 4 hours solution was crystallized. Crude material was vacuum filtrated and analyzed by XRPD. Arry-797: palmitic acid, Form P1 was obtained.

Further aspects and embodiments of the present disclosure are set out in the numbered clauses below:

A1. Crystalline polymorph of Arry-797.
A2. A crystalline polymorph of Arry-797 according to Clause A1 designated Form 1, which is characterized by data selected from one or more of the following:
a. an XRPD pattern having peaks at 6.2, 10.9, 11.4, 18.7 and 19.9 degrees 2-theta±0.2 degrees 2-theta;
b. an XRPD pattern as depicted in FIG. 1; and
c. combinations of these data.
   A3. A crystalline polymorph according to any of Clauses A1 or A2 designated Form 1, characterized by the XRPD pattern having peaks at 6.2, 10.9, 11.4, 18.7 and 19.9 degrees 2-theta±0.2 degrees 2-theta, and also having one, two, three, four or five additional peaks selected from 12.6, 13.1, 14.4, 22.2 and 24.8 degrees two theta±0.2 degrees two theta.
   A4. A crystalline polymorph according to any of Clauses A1, A2 or A3, designated Form 1, wherein the crystalline form is an anhydrous form.
   A5. A crystalline polymorph according to any of Clauses A1, A2, A3 or A4 designated Form 1, which contains: no more than about 20%, no more than about 10%, no more than about 5%, no more than about 2%, no more than about 1% or about 0% of any other crystalline forms of Arry-797.
   A6. A crystalline polymorph according to any of Clauses A1, A2, A3, A4, or A5, designated Form 1, which contains: no more than about 20%, no more than about 10%, no more than about 5%, no more than about 2%, no more than about 1% or about 0% of amorphous form of Arry-797.
   A7. A pharmaceutical composition comprising a crystalline polymorph according to any of Clauses A1, A2, A3, A4, A5 or A6 and at least one pharmaceutically acceptable excipient.
   A8. Use of a crystalline polymorph according to any of Clauses A1, A2, A3, A4, A5 or A6 for the preparation of a pharmaceutical composition and/or formulation, preferably wherein the pharmaceutical formulation is a tablet.
   A9. A process for preparing the pharmaceutical composition according to Clause A7, comprising combining a crystalline polymorph according to any of Clauses A1, A2, A3, A4, A5 or A6 with at least one pharmaceutically acceptable excipient.
   A10. A crystalline polymorph according to any of Clauses A1, A2, A3, A4, A5 or A6 or a pharmaceutical composition according to Clause A7 for use as a medicament.
   A11. A crystalline polymorph according to any of Claims A1, A2, A3, A4, A5 or A6 or a pharmaceutical composition according to Claim A7 for use in the treatment of LMNA-related DCM.
   A12. A method of treating LMNA-related DCM, comprising administering a therapeutically effective amount of a crystalline polymorph according to any of Clauses A1, A2, A3, A4, A5 or A6 or a pharmaceutical composition according to Clause A7 to a subject in need of the treatment.
   A13. Use of a crystalline polymorph according to any of Clauses A1, A2, A3, A4, A5 or A6 in the preparation of another solid state form of Arry-797.
   A14. A process for preparing a solid state form of Arry-797 comprising preparing any one or a combination of a crystalline polymorph according to any one of Clauses A1, A2, A3, A4, A5 or
   A6 and converting it to another solid state form thereof.
   B1. Crystalline Arry-797: maleic acid.
   B2. Crystalline Arry-797: maleic acid which is a co-crystal.
   B3. Crystalline Arry-797 maleate.
   B4. A crystalline polymorph according to any of Clause B1, B2 or B3 designated Form M1, which is characterized by data selected from one or more of the following:
a. an XRPD pattern having peaks at 3.7, 7.5, 9.2, 22.5 and 24.6 degrees 2-theta±0.2 degrees 2-theta;
b. an XRPD pattern as depicted in FIG. 2; and
c. combinations of these data.
   B5. A crystalline polymorph according to any of Clauses B1, B2, B3 or B4 designated Form M1, characterized by the XRPD pattern having peaks at 3.7, 7.5, 9.2, 22.5 and 24.6 degrees 2-theta±0.2 degrees 2-theta, and also having one, two, three or four additional peaks selected from 19.8, 21.9, 25.4 and 26.3 degrees two theta±0.2 degrees two theta.
   B6. A crystalline polymorph according to any of Clauses B1, B2, B3, B4 or B5 designated Form M1, which contains: no more than about 20%, no more than about 10%, no more than about 5%, no more than about 2%, no more than about 1% or about 0% of any other crystalline forms of Arry-797: maleic acid or crystalline Arry-797 maleate.
   B7. A crystalline polymorph according to any of Clauses B1, B2, B3, B4, B5, or B6, designated Form M1, which contains: no more than about 20%, no more than about 10%, no more than about 5%, no more than about 2%, no more than about 1% or about 0% of amorphous Arry-797: maleic acid or amorphous Arry-797 maleate.
   B8. A pharmaceutical composition comprising a crystalline polymorph according to any of Clauses B1, B2, B3, B4, B5, B6 or B7 and at least one pharmaceutically acceptable excipient.
   B9. Use of a crystalline polymorph according to any of Clauses B1, B2, B3, B4, B5, B6 or B7 for the preparation of a pharmaceutical composition and/or formulation, preferably wherein the pharmaceutical formulation is a tablet.
   B10. A process for preparing the pharmaceutical composition according to Clause B8, comprising combining a crystalline polymorph according to any of B1, B2, B3, B4, B5, B6 or B7 with at least one pharmaceutically acceptable excipient.
   B11. A crystalline polymorph according to any of Clauses B1, B2, B3, B4, B5, B6 or B7 or a pharmaceutical composition according to Clause B8 for use as a medicament.
   B12. A crystalline polymorph according to any of Claims B1, B2, B3, B4, B5, B6 or B7 or a pharmaceutical composition according to Claim B8 for use in the treatment of LMNA-related DCM.
   B13. A method of treating LMNA-related DCM, comprising administering a therapeutically effective amount of a crystalline polymorph according to any of B1, B2, B3, B4, B5, B6 or B7 or a pharmaceutical composition according to Clause B8 to a subject in need of the treatment.
   B14. Use of a crystalline polymorph according to any of Clauses B1, B2, B3, B4, B5, B6 or B7 in the preparation of another solid state form of Arry-797: maleic acid or Arry-797 maleate.
   B15. A process for preparing a solid state form of Arry-797: maleic acid or Arry-797 maleate comprising preparing any one or a combination of a crystalline polymorph according to any one of Clauses B1, B2, B3, B4, B5, B6 or B7 and converting it to another a solid state form thereof.
   C1. Crystalline Arry-797: fumaric acid.
   C2. Crystalline Arry-797: fumaric acid which is a co-crystal.
   C3. Crystalline Arry-797 fumarate.
   C4. A crystalline polymorph according to any of Clauses C1, C2 or C3 designated Form F1, which is characterized by data selected from one or more of the following:
a. an XRPD pattern having peaks at 5.3, 7.9, 10.5 and 16.9 degrees 2-theta±0.2 degrees 2-theta;
b. an XRPD pattern as depicted in FIG. 3; and
c. combinations of these data.
   C5. A crystalline polymorph according to any of Clauses C1, C2, C3 or C4 designated Form F1, characterized by the XRPD pattern having peaks at 5.3, 7.9, 10.5 and 16.9 degrees 2-theta±0.2 degrees 2-theta, and also having one, two, three or four additional peaks selected from 9.1, 11.5, 19.9 and 21.1 degrees two theta±0.2 degrees two theta.
   C6. A crystalline polymorph according to any of Clauses C1, C2, C3, C4 or C5 designated Form F1, which contains: no more than about 20%, no more than about 10%, no more than about 5%, no more than about 2%, no more than about 1% or about 0% of any other crystalline forms of Arry-797: fumaric acid or crystalline Arry-797 fumarate.
   C7. A crystalline polymorph according to any of Clauses C1, C2, C3, C4, C5, or C6 designated Form F1, which contains: no more than about 20%, no more than about 10%, no more than about 5%, no more than about 2%, no more than about 1% or about 0% of amorphous Arry-797: fumaric acid or amorphous Arry-797 fumarate.
   C8. A crystalline polymorph according to any of Clauses C1, C2 or C3 designated Form F2, which is characterized by data selected from one or more of the following:
a. an XRPD pattern having peaks at 6.6, 7.3, 13.3, 16.1, and 20.2 degrees 2-theta±0.2 degrees 2-theta;
b. an XRPD pattern as depicted in FIG. 4; and
c. combinations of these data.
   C9. A crystalline polymorph according to any of Clauses C1, C2, C3 or C8 designated Form F2, characterized by the XRPD pattern having peaks at 6.6, 7.3, 13.3, 16.1, and 20.2 degrees 2-theta±0.2 degrees 2-theta, and also having one, two, three, four or five additional peaks selected from 11.7, 15.3, 19.6, 22.0 and 24.5 degrees two theta±0.2 degrees two theta.
   C10. A crystalline polymorph according to any of Clauses C1, C2, C3, C8 or C9 designated Form F2, which contains: no more than about 20%, no more than about 10%, no more than about 5%, no more than about 2%, no more than about 1% or about 0% of any other crystalline forms of Arry-797: fumaric acid or crystalline Arry-797 fumarate.
   C11. A crystalline polymorph according to any of Clauses C1, C2, C3, C8, C9, or C10, designated Form F2, which contains: no more than about 20%, no more than about 10%, no more than about 5%, no more than about 2%, no more than about 1% or about 0% of amorphous Arry-797: fumaric acid or amorphous Arry-797 fumarate.
   C12. A pharmaceutical composition comprising a crystalline polymorph according to any of Clauses C1, C2, C3, C4, C5, C6, C7, C8, C9, C10 or C11 and at least one pharmaceutically acceptable excipient.
   C13. Use of a crystalline polymorph according to any of Clauses C1, C2, C3, C4, C5, C6, C7, C8, C9, C10 or C11 for the preparation of a pharmaceutical composition and/or formulation, preferably wherein the pharmaceutical formulation is a tablet.
   C14. A process for preparing the pharmaceutical composition according to Clause C12, comprising combining a crystalline polymorph according to any of C1, C2, C3, C4, C5, C6, C7, C8, C9, C10 or C11 with at least one pharmaceutically acceptable excipient.
   C15. A crystalline polymorph according to any of Clauses C1, C2, C3, C4, C5, C6, C7, C8, C9, C10 or C11 or a pharmaceutical composition according to Clause C12 for use as a medicament.
   C16. A crystalline polymorph according to any of Claims C1, C2, C3, C4, C5, C6, C7, C8, C9, C10 or C11 or a pharmaceutical composition according to Claim C12 for use in the treatment of LMNA-related DCM.
   C17. A method of treating LMNA-related DCM, comprising administering a therapeutically effective amount of a crystalline polymorph according to any of C1, C2, C3, C4, C5, C6, C7, C8, C9, C10 or C11 or a pharmaceutical composition according to Clause C12 to a subject in need of the treatment.
   C18. Use of a crystalline polymorph according to any of Clauses C1, C2, C3, C4, C5, C6, C7, C8, C9, C10 or C11 in the preparation of another solid state form of Arry-797: fumaric acid or Arry-797 fumarate.
   C19. A process for preparing a solid state form of Arry-797: fumaric acid or Arry-797 fumarate comprising preparing any one or a combination of a crystalline polymorph according to any one of Clauses C1, C2, C3, C4, C5, C6, C7, C8, C9, C10 or C11 and converting it to another a solid state form thereof.
   D1. Crystalline Arry-797: tartaric acid.
   D2. Crystalline Arry-797: tartaric acid which is a co-crystal.
   D3. Crystalline Arry-797 tartarate, D4. A crystalline polymorph according to any of Clauses D1, D2 or D3 designated Form T1, which is characterized by data selected from one or more of the following:
a. an XRPD pattern having peaks at 5.4, 16.4, 17.8 and 22.0 degrees 2-theta±0.2 degrees 2-theta;
b. an XRPD pattern as depicted in FIG. 5; and
c. combinations of these data.
   D5. A crystalline polymorph according to any of Clauses D1, D2, D3 or D4 designated Form T1, characterized by the XRPD pattern having peaks at 5.4, 16.4, 17.8 and 22.0 degrees 2-theta±0.2 degrees 2-theta, and also having one, two, three, four or five additional peaks selected from 12.6, 19.6, 21.0 and 26.5 degrees two theta±0.2 degrees two theta.
   D6. A crystalline polymorph according to any of Clauses D1, D2, D3, D4 or D5 designated Form T1, which contains: no more than about 20%, no more than about 10%, no more than about 5%, no more than about 2%, no more than about 1% or about 0% of any other crystalline forms of Arry-797: tartaric acid or crystalline Arry-797 tartarate.
   D7. A crystalline polymorph according to any of Clauses D1, D2, D3, D4, D5, or D6, designated Form T1, which contains: no more than about 20%, no more than about 10%, no more than about 5%, no more than about 2%, no more than about 1% or about 0% of amorphous Arry-797: tartaric acid or amorphous Arry-797 tartarate.
   D8. A pharmaceutical composition comprising a crystalline polymorph according to any of Clauses D1, D2, D3, D4, D5, D6 or D7 and at least one pharmaceutically acceptable excipient.
   D9. Use of a crystalline polymorph according to any of Clauses D1, D2, D3, D4, D5, D6 or D7 for the preparation of a pharmaceutical composition and/or formulation, preferably wherein the pharmaceutical formulation is a tablet.
   D10. A process for preparing the pharmaceutical composition according to Clause D8, comprising combining a crystalline polymorph according to any of D1, D2, D3, D4, D5, D6 or D7 with at least one pharmaceutically acceptable excipient.
   D11. A crystalline polymorph according to any of Clauses D1, D2, D3, D4, D5, D6 or D7 or a pharmaceutical composition according to Clause D8 for use as a medicament.
   D12. A crystalline polymorph according to any of Claims D1, D2, D3, D4, D5, D6 or D7 or a pharmaceutical composition according to Claim D8 for use in the treatment of LMNA-related DCM.
   D13. A method of treating LMNA-related DCM, comprising administering a therapeutically effective amount of a crystalline polymorph according to any of D1, D2, D3, D4, D5, D6 or D7 or a pharmaceutical composition according to Clause D8 to a subject in need of the treatment.
   D14. Use of a crystalline polymorph according to any of Clauses D1, D2, D3, D4, D5, D6 or D7 in the preparation of another solid state form of Arry-797: tartaric acid or Arry-797 tartarate.
   D15. A process for preparing a solid state form of Arry-797: tartaric acid or Arry-797 tartarate comprising preparing any one or a combination of a crystalline polymorph according to any one of Clauses D1, D2, D3, D4, D5, D6 or D7 and converting it to another a solid state form thereof.
   E1. Arry-797 HCl salt.
   E2. Crystalline polymorph of Arry-797 HCl salt.
   E3. A crystalline polymorph of Arry-797 HCl salt according to Clause E2 designated Form H1, which is characterized by data selected from one or more of the following:
a. an XRPD pattern having peaks at 4.0, 8.0, 12.0, 16.1 and 20.1 degrees 2-theta±0.2 degrees 2-theta;
b. an XRPD pattern as depicted in FIG. 6; and
c. combinations of these data.
   E4. A crystalline polymorph according to any of Clauses E2 or E3 designated Form H1, characterized by the XRPD pattern having peaks at 4.0, 8.0, 12.0, 16.1 and 20.1 degrees 2-theta±0.2 degrees 2-theta, and also having one, two, three, four or five additional peaks selected from 14.9, 17.1, 22.2, 24.2 and 32.4 degrees two theta±0.2 degrees two theta.
   E5. A crystalline polymorph according to any of Clauses E2, E3, or E4 designated Form H1, which contains: no more than about 20%, no more than about 10%, no more than about 5%, no more than about 2%, no more than about 1% or about 0% of any other crystalline forms of Arry-797.
   E6. A crystalline polymorph according to any of Clauses E2, E3, E4, or E5, designated Form H1, which contains: no more than about 20%, no more than about 10%, no more than about 5%, no more than about 2%, no more than about 1% or about 0% of amorphous form of Arry-797.
   E7. A pharmaceutical composition comprising a crystalline polymorph according to any of Clauses E2, E3, E4, E5 or E6 and at least one pharmaceutically acceptable excipient.
   E8. Use of a crystalline polymorph according to any of Clauses E2, E3, E4, E5 or E6 for the preparation of a pharmaceutical composition and/or formulation, preferably wherein the pharmaceutical formulation is a tablet.
   E9. A process for preparing the pharmaceutical composition according to Clause E7, comprising combining a crystalline polymorph according to any of Clauses E2, E3, E4, E5 or E6 with at least one pharmaceutically acceptable excipient.
   E10. A crystalline polymorph according to any of Clauses E2, E3, E4, E5 or E6 or a pharmaceutical composition according to Clause E7 for use as a medicament.
   E11. A crystalline polymorph according to any of Claims E2, E3, E4, E5 or E6 or a pharmaceutical composition according to Claim E7 for use in the treatment of LMNA-related DCM.
   E12. A method of treating LMNA-related DCM, comprising administering a therapeutically effective amount of a crystalline polymorph according to any of Clauses E1, E2, E3, E4, E5 or E6 or a pharmaceutical composition according to Clause E7 to a subject in need of the treatment.
   E13. Use of a crystalline polymorph according to any of Clauses E2, E3, E4, E5 or E6 in the preparation of another solid state form of Arry-797 or salts thereof.
   E14. A process for preparing a solid state form of Arry-797 or salts thereof comprising preparing any one or a combination of a crystalline polymorph according to any one of Clauses E2, E3, E4, E5 or E6 and converting it to another a solid state form thereof.
   F4. A crystalline polymorph according to Clause C3 designated Form F3, which is characterized by data selected from one or more of the following:
a. an XRPD pattern having peaks at 6.9, 12.3, 12.5 and 22.5 degrees 2-theta±0.2 degrees 2-theta;
b. an XRPD pattern as depicted in FIG. 7; and
c. combinations of these data.
   F5. A crystalline polymorph according to any of Clauses C3 or F4 designated Form F3, characterized by the XRPD pattern having peaks at 6.9, 12.3, 12.5 and 22.5 degrees 2-theta±0.2 degrees 2-theta, and also having one, two, three or four additional peaks selected from 7.1, 9.5, 17.5 and 20.7 degrees two theta±0.2 degrees two theta.
   F6. A crystalline polymorph according to any of Clauses C3, F4 or F5 designated Form F3, which contains: no more than about 20%, no more than about 10%, no more than about 5%, no more than about 2%, no more than about 1% or about 0% of any other crystalline forms of Arry-797: fumaric acid or crystalline Arry-797 fumarate.
   F7. A crystalline polymorph according to any of Clauses C3, F4, F5, or F6, designated Form F3, which contains: no more than about 20%, no more than about 10%, no more than about 5%, no more than about 2%, no more than about 1% or about 0% of amorphous Arry-797: fumaric acid or amorphous Arry-797 fumarate.
   G4. A crystalline polymorph according to Clause C3 designated Form F4, which is characterized by an XRPD pattern having peaks at 8.0, 9.3, and 12.0 degrees 2-theta±0.2 degrees 2-theta.
   G5. A crystalline polymorph according to any of Clauses C3 or G4 designated Form F4, which contains: no more than about 20%, no more than about 10%, no more than about 5%, no more than about 2%, no more than about 1% or about 0% of any other crystalline forms of Arry-797: fumaric acid or crystalline Arry-797 fumarate.
   G6. A crystalline polymorph according to any of Clauses C3, G4 or G5, designated Form F4, which contains: no more than about 20%, no more than about 10%, no more than about 5%, no more than about 2%, no more than about 1% or about 0% of amorphous Arry-797: fumaric acid or amorphous Arry-797 fumarate.
   H4. A crystalline polymorph according to Clause C3 designated Form F5, which is characterized by data selected from one or more of the following:
a. an XRPD pattern having peaks at 5.8, 8.7, 14.0, 17.4, 19.4 and 23.3 degrees 2-theta±0.2 degrees 2-theta;
b. an XRPD pattern as depicted in FIG. 9; and
c. combinations of these data.
   H5. A crystalline polymorph according to any of Clauses C3 or H4 designated Form F5, characterized by the XRPD pattern having peaks at 5.8, 8.7, 14.0, 17.4, 19.4 and 23.3 degrees 2-theta±0.2 degrees 2-theta, and also having one, two, three, four or five additional peaks selected from 11.6, 13.3, 15.5, 17.4 and 18.7 degrees two theta±0.2 degrees two theta.
   H6. A crystalline polymorph according to any of Clause C3 designated Form F5, which is characterized by data selected from one or more of the following:
a. an XRPD pattern having peaks at 5.8, 8.7, 14.0, 19.4 and 23.3 degrees 2-theta±0.2 degrees 2-theta;
b. an XRPD pattern as depicted in FIG. 9; and
c. combinations of these data.
   H7. A crystalline polymorph according to any of Clauses C3 or H6 designated Form F5, characterized by the XRPD pattern having peaks at 5.8, 8.7, 14.0, 19.4 and 23.3 degrees 2-theta±0.2 degrees 2-theta, and also having one, two, three, four or five additional peaks selected from 11.6, 13.3, 15.5, 17.4 and 18.7 degrees two theta±0.2 degrees two theta.
   H8. A crystalline polymorph according to any of Clauses C3, H4, H5, H6, or H7, designated Form F5, which contains: no more than about 20%, no more than about 10%, no more than about 5%, no more than about 2%, no more than about 1% or about 0% of any other crystalline forms of Arry-797: fumaric acid or crystalline Arry-797 fumarate.
   H9. A crystalline polymorph according to any of Clauses C3, H4, H5, H6, H7, or H8, designated Form F5, which contains: no more than about 20%, no more than about 10%, no more than about 5%, no more than about 2%, no more than about 1% or about 0% of amorphous Arry-797: fumaric acid or amorphous Arry-797 fumarate.
   I1. Crystalline Arry-797: palmitic acid.
   I2. Crystalline Arry-797: palmitic acid which is a co-crystal.
   I3. Crystalline Arry-797 palmitate.
   I4. A crystalline polymorph according to any of Clause I1, I2 or I3 designated Form P1, which is characterized by data selected from one or more of the following:
a. an XRPD pattern having peaks at 3.9, 6.7, 7.0, 7.5 and 10.6 degrees 2-theta±0.2 degrees 2-theta;
b. an XRPD pattern as depicted in FIG. 10; and
c. combinations of these data.
   I5. A crystalline polymorph according to any of Clauses I1, I2, I3 or I4 designated Form P1, characterized by the XRPD pattern having peaks at 3.9, 6.7, 7.0, 7.5 and 10.6 degrees 2-theta±0.2 degrees 2-theta, and also having one, two, three, four or five additional peaks selected from 9.6, 17.6, 20.5, 21.6 and 22.7 degrees two theta±0.2 degrees two theta.
   I6. A crystalline polymorph according to any of Clauses I1, I2, I3, I4 or I5 designated Form P1, which contains: no more than about 20%, no more than about 10%, no more than about 5%, no more than about 2%, no more than about 1% or about 0% of any other crystalline forms of Arry-797: palmitic acid or crystalline Arry-797 palmitate.
   I7. A crystalline polymorph according to any of Clauses I1, I2, I3, I4, I5, or I6, designated Form P1, which contains: no more than about 20%, no more than about 10%, no more than about 5%, no more than about 2%, no more than about 1% or about 0% of amorphous Arry-797: palmitic acid or amorphous Arry-797 palmitate.
   I8. A pharmaceutical composition comprising a crystalline polymorph according to any of Clauses I1, I2, I3, I4, I5, I6 or I7 and at least one pharmaceutically acceptable excipient.
   I9. Use of a crystalline polymorph according to any of Clauses I1, I2, I3, I4, I5, I6 or I7 for the preparation of a pharmaceutical composition and/or formulation, preferably wherein the pharmaceutical formulation is a tablet.
   I10. A process for preparing the pharmaceutical composition according to Clause I8, comprising combining a crystalline polymorph according to any of I1, I2, I3, I4, I5, I6 or I7 with at least one pharmaceutically acceptable excipient.
   I11. A crystalline polymorph according to any of Clauses I1, I2, I3, I4, I5, I6 or I7 or a pharmaceutical composition according to Clause 18 for use as a medicament.
   I12. A crystalline polymorph according to any of Claims I1, I2, I3, I4, I5, I6 or I7 or a pharmaceutical composition according to Claim 18 for use in the treatment of LMNA-related DCM.
   I13. A method of treating LMNA-related DCM, comprising administering a therapeutically effective amount of a crystalline polymorph according to any of I1, I2, I3, I4, I5, I6 or I7 or a pharmaceutical composition according to Clause 18 to a subject in need of the treatment.
   I14. Use of a crystalline polymorph according to any of Clauses I1, I2, I3, I4, I5, I6 or I7 in the preparation of another solid state form of Arry-797: palmitic acid or Arry-797 palmitate.
   I15. A process for preparing a solid state form of Arry-797: palmitic acid or Arry-797 palmitate comprising preparing any one or a combination of a crystalline polymorph according to any one of Clauses I1, I2, I3, I4, I5, I6 or I7 and converting it to another solid state form thereof.

1. (canceled)

2. A crystalline polymorph of Arry-797 designated Form 1, which is characterized by data selected from one or more of the following:
a. an XRPD pattern having peaks at 6.2, 10.9, 11.4, 18.7 and 19.9 degrees 2-theta±0.2 degrees 2-theta;
b. an XRPD pattern as depicted in FIG. 1; and
c. combinations of these data.

3. A crystalline polymorph according to claim 2 designated Form 1, characterized by the XRPD pattern having peaks at 6.2, 10.9, 11.4, 18.7 and 19.9 degrees 2-theta±0.2 degrees 2-theta, and also having one, two, three, four or five additional peaks selected from 12.6, 13.1, 14.4, 22.2 and 24.8 degrees two theta±0.2 degrees two theta.

4. A crystalline polymorph according to claim 2, designated Form 1, characterized by the XRPD pattern having peaks at 6.2, 10.9, 11.4, 12.6, 13.1, 14.4, 18.7, 19.9, and 22.2 degrees 2-theta±0.2 degrees 2-theta.

5. A crystalline polymorph according to claim 2, designated Form 1, wherein the crystalline form is an anhydrous form.

6. A crystalline polymorph according to claim 2, designated Form 1, which contains no more than about 20% of any other crystalline forms of Arry-797.

7. A crystalline polymorph according to claim 2, designated Form 1, which contains no more than about 20% of amorphous form of Arry-797.

8. (canceled)

9. A process for preparing a solid state form of Arry-797 comprising preparing any one or a combination of a crystalline polymorph according to claim 2, and converting it to another solid state form thereof.

10-31. (canceled)

32. A crystalline polymorph of Arry-797 tartrate designated Form T1, which is characterized by data selected from one or more of the following:
a. an XRPD pattern having peaks at 5.4, 16.4, 17.8 and 22.0 degrees 2-theta±0.2 degrees 2-theta;
b. an XRPD pattern as depicted in FIG. 5; and
c. combinations of these data.

33. A crystalline polymorph according to claim 32, designated Form T1, characterized by the XRPD pattern having peaks at 5.4, 16.4, 17.8 and 22.0 degrees 2-theta±0.2 degrees 2-theta, and also having one, two, three, four or five additional peaks selected from 12.6, 19.6, 21.0 and 26.5 degrees two theta±0.2 degrees two theta.

34. A crystalline polymorph according to claim 32, designated Form T1, characterized by the XRPD pattern having peaks at 5.4, 12.6, 16.4, 17.8, 19.6, 21.0, 22.0, and 26.5 degrees 2-theta±0.2 degrees 2-theta.

35. A crystalline polymorph according to claim 32, wherein the crystalline form is an anhydrous form.

36. A crystalline polymorph according to claim 32, designated Form T1, which contains no more than about 20% of any other crystalline forms of Arry-797 tartrate.

37. A crystalline polymorph according to claim 32, designated Form T1, which contains no more than about 20% of amorphous Arry-797 tartrate.

38. (canceled)

39. A process for preparing a solid state form of Arry-797 tartrate comprising preparing any one or a combination of a crystalline polymorph according to claim 32, and converting it to another a solid state form thereof.

40-49. (canceled)

50. A crystalline polymorph of Arry-797 HCl salt designated Form H1, which is characterized by data selected from one or more of the following:
a. an XRPD pattern having peaks at 4.0, 8.0, 12.0, 16.1 and 20.1 degrees 2-theta±0.2 degrees 2-theta;
b. an XRPD pattern as depicted in FIG. 6; and
c. combinations of these data.

51. A crystalline polymorph according to claim 50, designated Form H1, characterized by the XRPD pattern having peaks at 4.0, 8.0, 12.0, 16.1 and 20.1 degrees 2-theta±0.2 degrees 2-theta, and also having one, two, three, four or five additional peaks selected from 14.9, 17.1, 22.2, 24.2 and 32.4 degrees two theta±0.2 degrees two theta.

52. A crystalline polymorph according to claim 50, designated Form H1, characterized by the XRPD pattern having peaks at 4.0, 8.0, 12.0, 14.9, 16.1, 17.1, 20.1, 22.2, and 24.2 degrees 2-theta±0.2 degrees 2-theta.

53. A crystalline polymorph according to claim 50, wherein the crystalline form is an anhydrous form.

54. A crystalline polymorph according to claim 50, designated Form H1, which contains no more than about 20% of any other crystalline forms of Arry-797 HCl salt.

55. A crystalline polymorph according to claim 50, designated Form H1, which contains no more than about 20% of amorphous form of Arry-797 HCl salt.

56. (canceled)

57. A process for preparing a solid state form of Arry-797 or salts thereof comprising preparing any one or a combination of a crystalline polymorph according to claim 50, and converting it to another a solid state form thereof.

58. A pharmaceutical composition comprising a crystalline polymorph according to claim 2, and at least one pharmaceutically acceptable excipient.

59. (canceled)

60. A process for preparing the pharmaceutical composition according to claim 58, comprising combining the crystalline polymorph with at least one pharmaceutically acceptable excipient.

61. (canceled)

62. (canceled)

63. A method of treating LMNA-related DCM, comprising administering a therapeutically effective amount of a crystalline polymorph according to claim 2 to a subject in need of the treatment.
